(19) United States
(12) Patent Application Publication
(10) Pub. No.: US 2023/0310182 A1
(43) Pub. Date: Oct. 5, 2023

(54) METHOD FOR CONTROLLING A PROSTHESIS OR ORTHESIS

(71) Applicant: Otto Bock Healthcare Products GmbH, Vienna (AT)

(72) Inventors: Thomas Hofmann, Vienna (AT); Dirk Seifert, Vienna (AT)

(21) Appl. No.: 18/016,831

(22) PCT Filed: Jul. 20, 2021
(86) PCT No.: PCT/EP2021/070284

§ 371 Date: Jan. 18, 2023

(30) Foreign Application Priority Data
Jul. 20, 2020 (DE) 10 2020 004 338.0

(51) Int. Cl. A61F 2/68; A61F 2/64

(52) U.S. Cl. CPC A61F 2/68; A61F 2/64; A61F 2002/7625

(57) ABSTRACT

The invention relates to a method for controlling a prosthesis or orthesis of the lower extremity, the prosthesis or orthesis comprising an upper part (10) and a lower part (20) which is connected to the upper part (10) via a knee joint and is mounted so as to be pivotable relative to the upper part (10) about a joint pin (15); wherein an adjustable resistance device (40) is situated between the upper part (10) and the lower part (20), by means of which resistance device a resistance (Rf) is modified on the basis of sensor data; wherein state information is detected by sensors, a cyclical movement different from walking is determined and the resistance (Rf) is adjusted to a low level during the cyclical movement; wherein determining the cyclical movement comprises the following steps: a. detecting the flexion angle (α_{K}) and at least one absolute angle (αS) of the lower part (20) and/or the upper part (10) over at least one movement cycle, b. identifying the cyclical movement from the relative movement of the upper part (10) and the lower part (20) and the absolute movements of the upper part (10) and/or the lower part (20) in space.

The invention relates to a method for controlling a prosthesis or orthosis of the lower extremity, the prosthesis or orthosis having an upper part and a lower part which is connected to the upper part via a knee joint and is mounted so as to be pivotable relative to the upper part about a joint axis, wherein an adjustable resistance device is arranged between the upper part and the lower part, by means of which resistance device a resistance against knee flexion and/or extension is modified on the basis of sensor data, wherein state information is detected via sensors, a cyclical movement different than walking is determined, and the resistance is adjusted to a low level during the cyclical movement.

Artificial knee joints are used in prostheses and orthoses, and also in exoskeletons as a special case of orthoses. An artificial knee joint has an upper part and a lower part which are mounted so as to be pivotable relative to each other about a joint axis, the knee axis. In the simplest case, the knee joint is in the form of a single-axis knee joint, in which, for example, a pin or two bearing points arranged on a pivot axis form a single knee axis. Also known are artificial knee joints which do not form a fixed axis of rotation between upper part and lower part, but have either sliding or rolling surfaces or a multiplicity of link bars connected to one another in an articulated manner. In order to be able to influence the movement properties of the knee joints and to obtain a movement behavior of the orthosis or prosthesis or exoskeleton that emulates the natural gait behavior, resistance devices via which the resistance can be changed are provided between the upper part and the lower part. Purely passive resistance devices are passive dampers, for example hydraulic dampers, pneumatic dampers, or dampers that change the movement resistance on the basis of magnetorheological effects. There are also active resistance devices, for example motors or other drives, which, via a corresponding connection, can be operated as generators or energy stores.

The knee joints, that is to say the prosthetic joints or orthotic knee joints, are fastened to the patient by attachment means. In the case of prosthetic knee joints, fastening generally takes place by way of a thigh socket, which receives a limb stump. Alternative types of fastening are likewise possible, for example with osseointegrated attachment means or by means of belts and other devices. In the case of orthoses and exoskeletons, the upper part and lower part are fastened directly to the thigh and the lower leg. The fastening devices provided for this purpose are, for example, belts, sleeves, cups or frame structures. Orthoses can also have foot parts for supporting a foot or shoe. The foot parts can be mounted in an articulated manner on the lower part.

An artificial knee joint with the maximum extension that is structurally achievable has a knee angle of 180°; a hyperextension, that is to say an angle of more than 180°, on the posterior side is generally not provided. Pivoting of the lower part posteriorly relative to the upper part is referred to as knee flexion, while pivoting anteriorly or in a forward direction is referred to as extension.

EP 2 498 729 B1 discloses a method for controlling an artificial orthotic or prosthetic joint of a lower extremity, having a resistance device which is assigned at least one actuator via which the flexion resistance and/or extension resistance is modified depending on sensor data, wherein state information is made available via sensors while the joint is being used. If a cyclical movement different than walking is determined, the resistance is reduced for the duration of the cyclical movement. In order to determine the cyclical movement, the time intervals between the maxima and minima of the knee angle and the characteristic knee angle profiles are used. If no maxima and minima are detected, the resistance is increased.

A problem here is that, when riding a bicycle, situations can arise in which the user does not pedal evenly. For example when cycling downhill or when the user has to stand up in order to drive over an obstacle.

The object of the present invention is to make available a method by which cycling with an artificial knee joint is made easier.

According to the invention, this object is achieved by a method having the features of the main claim.

Advantageous refinements and developments of the invention are disclosed in the dependent claims, the description and the figures.

In the method for controlling a prosthesis or orthosis of the lower extremity, the prosthesis or orthosis having an upper part and a lower part which is connected to the upper part via a knee joint and is mounted so as to be pivotable relative to the upper part about a joint axis, wherein an adjustable resistance device is arranged between the upper part and the lower part, by means of which resistance device a resistance is modified on the basis of sensor data, wherein state information is detected via sensors, a cyclical movement different than walking is determined, and the resistance is adjusted to a low level during the cyclical movement, provision is made that the determination of the cyclical movement comprises detecting the flexion angle and at least one absolute angle of the lower part and/or of the upper part over at least one movement cycle, and identifying the cyclical movement from the relative movement of upper part and lower part and from the absolute movements of upper part and/or lower part in space. The flexion angle does not have to be measured directly using an angle sensor; it can also be calculated using the absolute angles of the upper and lower part. By detecting the flexion angle, i.e. the relative pivoting between the upper part and the lower part, starting from an extended position with a knee angle of 180°, and by detecting at least one absolute angle of the upper part or the lower part via at least one inertial angle sensor or IMU, it is possible to detect the movement of upper part and lower part relative to each other and also the movement in space. The permanent detection of both the flexion angle and one or more absolute angles, i.e. the absolute angle of the upper part and/or the absolute angle of the lower part, enables a simple switchover to the bicycle function of the artificial knee joint, without the user of the orthosis or prosthesis having to perform any additional activity. There is no requirement to change a switch position, activate an app or carry out a special movement pattern; instead, the switchover to the bicycle function is triggered solely by the pedaling movement. The bicycle function is maintained until at least one termination criterion is detected. A continuous, uninterrupted cyclical movement, which differs in particular from walking, does not have to be present. If, for example, the prosthesis or orthosis is placed on the ground when dismounting, the bicycle function is deactivated; during continuous cycling, even without cyclical movements, for example when the user is riding while standing on the pedals, the function is advantageously not deactivated, so that a complete detection cycle does not have to be run through again in order to switch to a bicycle function. After detection of the cyclical movement, the extension resistance and the flexion resistance are advantageously reduced for the bicycle function. Where resistance is mentioned hereinbelow in general, this means flexion resistance and extension resistance individually or together. If extension resistance or flexion resistance is explicitly mentioned, only this resistance is meant.

In a development of the invention, provision is made that the reduction of the resistance takes place only when a flexion angle change, in particular the amount of the flexion angle change, added up over an interval in which certain conditions are met, is greater than a specified limit value, in particular greater than 240°. In order to detect a cyclical pedaling movement and thereby activate the bicycle function, it is advantageous if both the flexion angle and the absolute angles of the upper part and/or of the lower part meet certain criteria, and indeed over a certain period of time, so that the switch-on command takes place. Advantageously, this period of time is determined via the flexion angle change. The adding-up of the amount of the flexion angle has the advantage that the flexion angle parameter can be detected continuously and no distinction has to be made between flexion and extension. If the flexion angle were added up with a sign, the sum would be zero after one pedal revolution and a cyclical movement could never be determined, since this requires a repetition. Alternatively, the flexion angle changes are only added up at certain time intervals or under certain conditions, e.g. only with an increasing flexion angle or only with a decreasing flexion angle or with a change only under a certain axial load on the lower part or with a simultaneous change in the angle of a foot part to the lower part. The flexion angle is monitored, and its change is accumulated or added up at certain time intervals. If the necessary conditions for detection of the cyclical movement and activation of the cycling mode are present over the period of the cumulative absolute flexion angle changes, the resistance is reduced.

If a condition is no longer met over the period of time of determination of the cyclical movement, for example if the flexion angle is not within the specified limit values or if the lower part and/or the upper part is displaced in such a way that they are not within the specified parameters, the counter for the flexion angle is reset. This prevents a situation where random movements or matches to the parameters are added up throughout the day. The limit values or parameters must be met over the defined period of time, for example the sum of the flexion angle change or the cumulative absolute flexion angle change. If the necessary conditions are interrupted before the bicycle mode is activated, the resistance is not reduced and the riding mode is not switched to.

Provision is preferably made for the resistance to be reduced only when the established conditions for the flexion angle and the inclination angle of the lower part are present or fulfilled over the cumulative flexion angle change, e.g. the sum of the amount of the flexion angle change greater than 240°. The necessary conditions for a reduction in resistance must be present over the entire period of the cumulative flexion angle change, e.g. greater than 240°, otherwise the bicycle function is not activated. Once the bicycle function has been activated, i.e. the necessary conditions are present over a specified period of time of a flexion angle change, in particular greater than 240°, it is no longer necessary to perform the uniform, cyclical movement in order to continue to have the reduced resistance. The reduced resistance is maintained until defined changes occur or sensor values are detected that make an increase in the flexion resistance and/or extension resistance necessary.

In a development of the invention, provision is made that a cyclical movement is ascertained only if the flexion angle is greater than a specified limit value, in particular greater than 10°, in particular greater than 15°. A flexion angle greater than 10°, in particular greater than 15°, is a good indicator of a pedaling movement being performed. When riding a bicycle, at least when seated, a flexion angle is or should always be greater than 10°, in particular greater than 15°, at the beginning. If this is the case, it can be assumed with a high degree of certainty that there is a pedaling movement.

In a development of the invention, provision is made that a cyclical movement is ascertained only if the angle of inclination of the lower part and/or the flexion angle does not exceed and/or fall below certain limit values. The angle of inclination of the lower part in space and its profile over time are a good indicator of whether or not a cyclical movement is present and whether or not the cyclical movement is maintained. In combination with the flexion angle, those parameters that are meaningful for the presence or absence of a cyclical movement can be narrowed down. If the angle of inclination of the lower part is within certain limit values for a specified period of time, i.e. does not exceed or fall below the limit values, and if the same also applies to the flexion angle, conclusions can be drawn as regards the presence or absence of the cyclical movement. If the presence of the cyclical movement is detected, the resistance is reduced; if the bicycle mode is not to be switched on or activated, the resistance remains at the initial level, for example the stance phase level for walking on level ground.

In a development of the invention, provision is made that a cyclical movement is ascertained only if the angle of inclination of the lower part relative to the vertical does not exceed a limit value, in particular is less than 5°. The angle of inclination of the lower part, the so-called roll angle, is also an indicator of a cycling activity being carried out. If the angle of inclination of the lower part forward relative to the vertical over a certain period of time is always less than a limit value, in particular less than 5°, where a further forward inclination is harmless, it can be assumed that the movement is a cycling movement. The rearward inclination of the lower part, when the foot part or the ankle joint axis is situated in front of the knee joint axis, is assumed to be a positive roll angle. A slight rearward inclination can be tolerated and occurs when riding a bicycle. The limit value should not be exceeded, and in particular should not be greater than 5°.

In a development of the invention, provision is made that the end of the cyclical movement is identified from the fact that the lower part is relieved of an axial load or axial force for a predetermined period of time, in particular greater than >150 ms, and the flexion angle falls below a limit value, in particular less than 15°, or the inclination of the lower part to the vertical exceeds a limit value, in particular greater than 10°. The lower part can be relieved of an axial force when the axial force acting in the direction of the knee joint falls below a specified limit value or the lower part is completely relieved, i.e. no axial force acts in the direction of the knee joint. If the lower part is relieved for a predetermined period of time and no axial force acting in the direction of the joint axis acts on the lower part, this can serve as a reference value for the fact that the user has taken his foot off the pedal and may thus want to dismount or has set his foot down or has to set his foot down in order to support himself elsewhere. If the flexion angle also falls below a limit value, in particular less than 15°, such that an extension of the fitted leg can be assumed, or if the inclination of the lower part to the vertical exceeds a limit value, in particular greater than 10°, which occurs for example when dismounting and swinging the leg over the back wheel and the saddle, cycling is advantageously identified as being finished and in particular the flexion resistance is increased again. The cyclical movement does not have to be carried out continuously from the outset, only over a certain period of time, namely over a certain number of pedal revolutions or over a certain sum of a flexion angle change in order to set the reduced resistance.

In a development of the invention, provision is made that, when the cyclical movement is interrupted while the lower part is simultaneously subjected to an axial force, an extension of the knee joint below a specified flexion angle, in particular below 10°, is prevented and the flexion resistance increased and, after an end stop is reached, followed by flexion and renewed extension, the resistance is set anew for the cyclical movement. If, for example, a knee extension is detected, which represents an interruption in the cyclical movement, but not an interruption in cycling and thus an interruption in the resistance mode set in the resistance device, and at the same time an axial force is detected, an extension of the knee joint beyond a certain value is prevented, for example over an extension value of less than 10°, in order to prevent the knee joint from being fully extended. Full extension when the pedaling movement is interrupted, for example when standing up, can make flexion of the knee joint more difficult, which is undesirable if the pedaling movement is to be resumed. At the same time, the flexion resistance is increased, for example to a stance phase level when walking, which enables basic flexion. As a result, for sitting down after standing up, sufficient flexion damping or a flexion resistance is provided in order to allow controlled knee flexion. If the end stop of the extension is reached and a subsequent flexion takes place against the then possibly increased resistance, the resistance setting for the cyclical movement is set again with a movement reversal and renewed extension, i.e. the flexion resistance and possibly the extension resistance are reduced. This prevents the uniformity criteria with regard to the flexion angle and the inclination angle of the lower part from having to be present again over the complete necessary value of the cumulative, absolute flexion angle change, for example the amount of the flexion angle change. For example, the situation of standing up, for example when riding over an obstacle, is detected and a simplified activation of the resistance device is made possible.

In a development of the invention, provision is made that, in order to determine the cyclical movement, the angular velocities are calculated, and the quotient of the angular velocities is determined in specified time segments. Changes in the quotient of the angular velocities are determined as tangent slopes in specified time segments. A determination of the cyclical movement takes place when the time profile of the tangent slopes is monotonically increasing. A monotonically increasing time profile of the tangent slope is also present when there are smaller deviations from the monotony condition. It is essential that the change in slope does not fall below a specified limit value. The calculation of the angular velocities, i.e. the angular velocities of the flexion angle and of the angle of inclination of the lower part, and also the calculation of the quotient of the angular velocities in specified time segments, makes it possible to determine the tangent slopes of the profile of the two angular velocities which can be represented as a closed, convex two-dimensional curve. The determination of the cyclical movement and thus of a reduction in the resistance can be assumed if the tangent slope is found to be monotonically increasing over a complete circuit of the curve.

In a development of the invention, provision is made that an evaluation of the angular velocities takes place only when a limit value of the flexion angular velocity is exceeded. The consideration of the flexion angular velocity gives an additional indication that a cycling movement is actually taking place and that it is not small recurring movements, for example when sitting, that are mistakenly being identified as a cycling movement. In addition, when determining the tangent slope, the consideration of points of discontinuity is thereby avoided.

To determine the cyclical movement, the phase space of two angular parameters and/or their derivatives can be used. In particular, the direction of rotation in the phase space can be determined and used to identify whether there is a cyclical movement different than walking on the level and a corresponding reduction in resistance is performed. The angle parameters are the flexion angle and at least one absolute angle of one of the components around the knee joint. The angles in space are used for the lower part and/or the upper part.

In a development of the invention, provision is made that the trajectory of the foot part and/or its derivatives relative to a determined hip rotation point are/is determined from the knee angle and/or absolute angles of upper part and lower part and used for the determination of the cyclical movement. From the knee angle, the absolute angles or an absolute angle and from the known geometric relationships between the knee axis and the foot part, for example a reference point of the foot part near the sole of the foot or the position of the ankle joint axis, it is possible to determine the trajectory of the foot part. The trajectory or its time derivatives can be related to a determined rotation point. The hip rotation point is determined in any case by an orthopedic technician during the adjustment of the orthosis or prosthesis. The distance to the knee joint axis is therefore also known. The course of the trajectory, the trajectory speeds and/or trajectory accelerations of a reference point of a foot part to the rotation point can then be determined from the known geometric relationships and angle changes and can serve as an indicator of whether a cyclical movement is present or not.

An exemplary embodiment of the invention is discussed in more detail below with reference to the figures, in which:

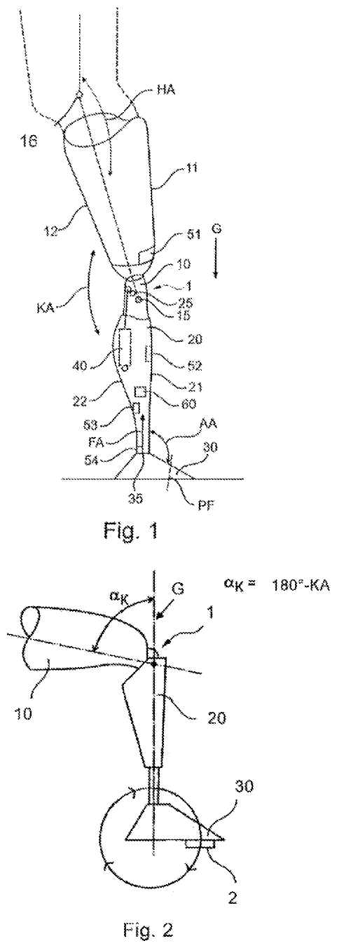

FIG. 1 shows a schematic illustration of a prosthetic leg;

FIG. 2 shows a schematic illustration of a pedaling movement;

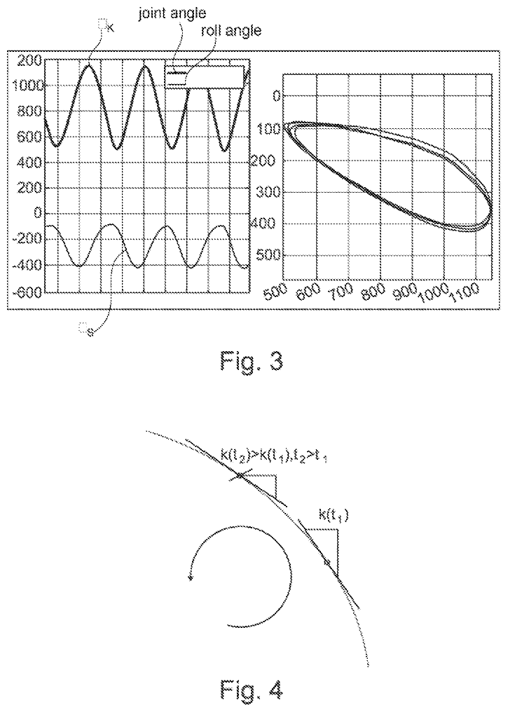

FIG. 3 shows a common illustration of a flexion angle and of a roll angle;

FIG. 4 shows a schematic illustration of the calculation of a tangent slope;

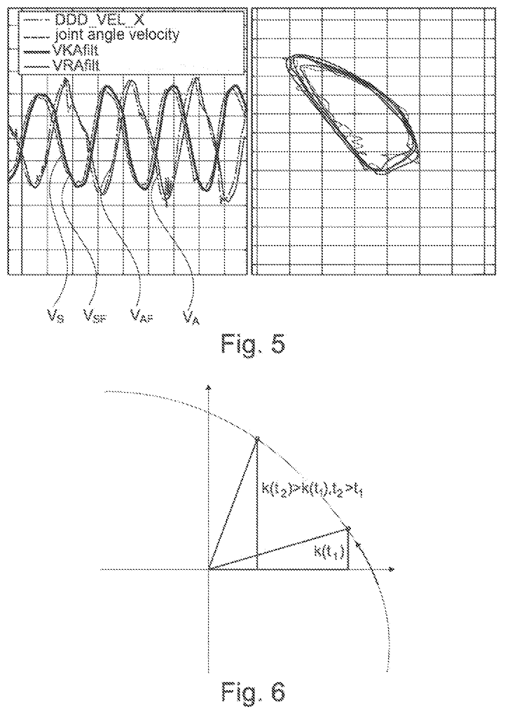

FIG. 5 shows a comparison of measured and calculated angular velocities;

FIG. 6 shows the profile of the slope of a connection line in the diagram according to FIG. 5;

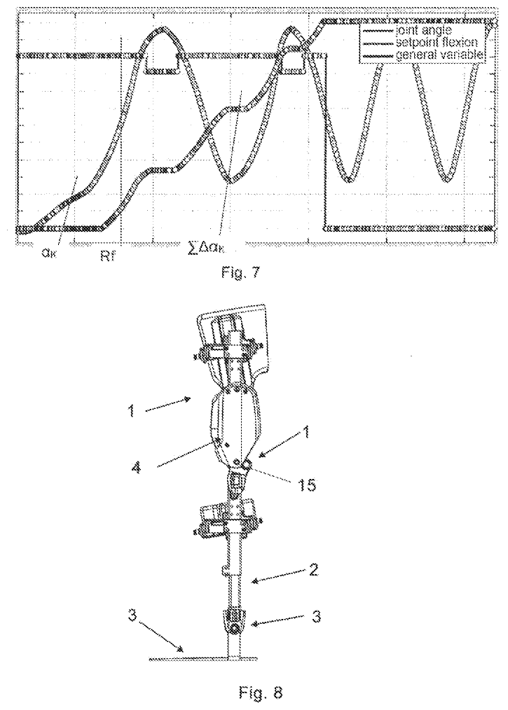

FIG. 7 shows a data record for setting off and the activation of a cycling function, and

FIG. 8 shows an illustration of an orthosis.

FIG. 1 shows a schematic illustration of an artificial knee joint 1 in an application on a prosthetic leg. As an alternative to an application on a prosthetic leg, a correspondingly designed artificial knee joint 1 can also be used in an orthosis or an exoskeleton. Instead of replacing a natural joint, the artificial knee joint is then arranged medially and/or laterally on the natural joint. In the exemplary embodiment shown, the artificial knee joint 1 is in the form of a prosthetic knee joint having an upper part 10 with a side 11, which is anterior or situated to the front in the walking direction, and a posterior side 12, which is located opposite the anterior side 11. A lower part 20 is arranged on the upper part 10 so as to be pivotable about a pivot axis 15. The lower part 20 also has an anterior side 21 or front side and a posterior side 22 or rear side. In the exemplary embodiment shown, the knee joint 1 is designed as a monocentric knee joint; it is in principle also possible to control a polycentric knee joint in a corresponding manner. At the distal end of the lower part 20 there is arranged a foot part 30 which can be connected to the lower part either in the form of a rigid foot part 30 with a fixed foot joint or by a pivot axis 35, in order to make possible a movement sequence that emulates the natural movement sequence.

Between the posterior side 12 of the upper part 10 and the posterior side 22 of the lower part 20, the knee angle KA is measured. The knee angle KA can be measured directly by means of a knee angle sensor 25, which can be arranged in the region of the pivot axis 15. The knee angle sensor 25 can be coupled to a torque sensor or can have such a sensor, in order to detect a knee moment about the joint axis 15. On the upper part 10 there is arranged an inertial angle sensor or an IMU 51, which measures the spatial position of the upper part 10, for example in relation to a constant force direction, for example the gravitational force G, which is directed vertically downward. An inertial angle sensor or an IMU 53 is likewise arranged on the lower part 20 in order to determine the spatial position of the lower part while the prosthetic leg is in use.

In addition to the inertial angle sensor 53, an acceleration sensor and/or transverse force sensor 53 can be arranged on the lower part 20 or on the foot part 30. By means of a force sensor or torque sensor 54 on the lower part 20 or foot part 30, an axial force FA acting on the lower part 20 or an ankle moment acting about the ankle joint axis 35 can be determined.

Between the upper part 10 and the lower part 20 there is arranged a resistance device 40, in order to influence a pivoting movement of the lower part 20 relative to the upper part 10. The resistance device 40 can be in the form of a passive damper, in the form of a drive, or in the form of what is called a semi-active actuator with which it is possible to store movement energy and deliberately release it again at a later time in order to slow or assist movements. The resistance device 40 can be in the form of a linear or rotary resistance device. The resistance device 40 is connected to a control device 60, for example in a wired manner or via a wireless connection, which in turn is coupled to at least one of the sensors 25, 51, 52, 53, 54. The control device 60 electronically processes the signals transmitted by the sensors, using processors, computing units or computers. It has an electrical power supply and at least one memory unit in which programs and data are stored and in which a working memory for processing data is provided. After the sensor data have been processed, an activation or deactivation command is output, with which the resistance device 40 is activated or deactivated. By activation of an actuator in the resistance device 40 it is possible, for example, to open or close a valve or to generate a magnetic field, in order to change a damping behavior.

To the upper part 10 of the prosthetic knee joint 1 there is fastened a prosthesis socket, which serves to receive a thigh stump. The prosthetic leg is connected to the hip joint 16 by way of the thigh stump. On the anterior side of the upper part 10, a hip angle HA is measured, which is marked on the anterior side 11 between a vertical line through the hip joint 16 and the longitudinal extent of the upper part 10 and the connecting line between the hip joint 16 and the knee joint axis 15. If the thigh stump is lifted and the hip joint 16 flexed, the hip angle HA decreases, for example when sitting down. Conversely, the hip angle HA increases in the case of an extension, for example when standing up or in the case of similar movement sequences, for example when pushing down on a pedal during cycling.

The prosthetic leg with the artificial knee joint between the upper part 10 and the lower part 20 is shown schematically in FIG. 2. The prosthetic foot 30 is placed on a pedal 2 and performs a circular movement along the pedal path. In the position shown, the lower part 20, or the longitudinal extent of the lower part 20, is located in a vertical position, the inclination of the lower part 20 to the vertical G is therefore 0°, and the roll angle is therefore 0. The flexion angle α_{K}, which results from the pivoting of the upper part 10 relative to the lower part 20 about the knee axis, is the change to the fully extended or straightened prosthetic leg. The flexion angle α_{K} is calculated from the difference between 180° and the knee angle KA. When performing a pedaling movement, as is necessary when riding a bicycle to propel the bicycle, cyclical movements are performed. Forces from the foot part 30 to the pedals 20 are introduced at the force application point PF, which is shown in FIG. 1. As a rule, these are compressive forces, since it is only with difficulty that a prosthesis user or an orthosis user with limited motor skills in the muscles can apply forces that act against the direction of gravity. Due to the lack of muscle connections to the lower part 20, no forces that act in the horizontal direction can generally be applied to the pedals 2. Since, for safety reasons, the foot part 30 is not fixed on the pedal 2, no forces acting against the direction of gravity, for example by activation of the hip flexor muscle, are transmitted. During the pedaling movement, flexion resistances and extension resistances are therefore undesirable, since these oppose a rotary movement of the pedals 2 about the axis of rotation of the pedal crank.

Provision is therefore made that at least the resistance, in particular flexion resistance and possibly also an extension resistance, is reduced depending on the detection of a cyclical movement when riding a bicycle. This detection and reduction of the resistance is to be carried out as quickly as possible without the user of the artificial knee joint having to take any further measures apart from carrying out the cycling movement. Once the cyclical movement sequence associated with riding a bicycle is detected, the cycling mode remains set until a change in parameters or sensor values is detected that indicates cessation of cycling, which may not necessarily be accompanied by cessation of the cyclical movement.

FIG. 3 shows the flexion angle α_{K} and the roll angle αₛ as the angle of inclination of the lower part 20 in space. The illustration on the left shows that both angles run substantially cyclically during cycling and run through a uniform change at a constant speed of the pedals. The illustration on the right shows both values in an X-Y diagram. The pedal movement results in a closed, convex, two-dimensional curve, with the flexion angle α_{K} being plotted on the x-axis and the lower-leg angle αₛ on the y-axis. If a uniform and cyclical movement has been detected, specifically over a specified period of time, for example over two pedal revolutions, the resistance of the resistance device is reduced. When riding a bicycle, the curve in the right-hand illustration of FIG. 3 is traversed counterclockwise, the time profile of the tangent slope of the curve, as illustrated in FIG. 4, being read to the right and therefore increasing monotonically. The slope k₁ at a specific point in time t₁ is calculated from the quotient of the horizontal component and the vertical component; the slope k₂ of the tangent at a later point in time t₂ is greater in the illustrated exemplary embodiment. The tangent slope is the quotient of the changes in the X and Y components, i.e. the quotient of the angular velocities of the flexion angle as and the inclination angle as of the lower part 20.

A variant of the calculation or determination of the tangent slopes is shown in FIGS. 5 and 6. FIG. 5 shows the angular velocities both of the measurements and of filtered measurements. The angular velocity V_{S} of the lower part and the angular velocity V_{A} of the flexion angle have interference signals, for example due to vibrations or measurement errors, which are identified by the uneven curve progressions. The filtered angular velocity signals V_{SF} and V_{AF} are also plotted. In the right-hand illustration, the values are shown in the X-Y diagram with the origin of the coordinates at the cross. Here too, the differences between the comparatively smooth curves and the uneven curves are clear. The slope of the connection line between the origin X and the respective point on the curve in the X-Y diagram at time t₁ and time t₂ is calculated using the rule according to FIG. 4. At a knee angular velocity V_{A}=0, the sign would jump from plus infinity to minus infinity. Such a value is excluded from the analysis since the evaluation only takes place when a limit value for the flexion angle velocity is exceeded. In principle, smaller deviations from the monotony condition, as explained above, may also be permissible in order to continue to regard the uniformity criterion as fulfilled.

In order to identify whether the cyclical movement of cycling is being carried out, the amount of the flexion angle changes ΣΔα_{K} is added up, the flexion angle and the roll angle and/or the orientation of the upper part in space being considered. The course of the flexion angle α_{K} at the start of cycling is recorded in the diagram in FIG. 7. Starting with an extended leg, flexion with subsequent extension and again flexion and extension is carried out. The resistance Rf initially remains at a high level. The count value ΣΔα_{K} is always increased when there is a sufficient absolute knee angle velocity, i.e. a sufficiently large change in the flexion angle α_{K}. In the region of movement reversal with a reduced change in the flexion angle, the count value is not updated, in order not to jeopardize the sufficient safety in identifying the cycling movement. The same applies to the run after calculating a maximum extension, i.e. a lower relative minimum of the flexion angle α_{K}. If the counter reaches the specified limit value, for example a change in the flexion angle of 240°, which can be seen from the horizontal progression of the counter ΣΔα_{K}, the resistance Rf is reduced to the desired value, for example dropped to almost 0. The counter value is not updated any further, since the uniformity criterion is sufficiently met.

FIG. 8 shows a schematic illustration of an exemplary embodiment of an orthosis with an upper part 10 and a lower part 20 mounted thereon so that it can pivot about a pivot axis 15, with which orthosis the method can also be carried out. An artificial knee joint 1 is formed between the upper part 10 and the lower part 20 and is arranged laterally with respect to a natural knee joint in the illustrated exemplary embodiment. In addition to an arrangement of upper part 10 and lower part 20 on one side relative to a leg, two upper parts and lower parts can also be arranged medially and laterally with respect to a natural leg. At its distal end, the lower part 20 has a foot part 30 which is pivotable about an ankle joint axis 35 with respect to the lower part 20. The foot part 30 has a foot plate on which a foot or shoe can be placed. Fastening devices for securing to the lower leg and thigh are arranged on the lower part 20 and on the upper part 30, respectively. Devices for securing the foot on the foot part 30 can also be arranged on the foot part 30. The fastening devices can be in the form of buckles, straps, clasps or the like, in order to be able to fit the orthosis on the user's leg in a releasable manner and to remove it again without destroying it. The resistance device 40 is fastened to the upper part 10, is supported on the lower part 20 and on the upper part 10 and provides an adjustable resistance to pivoting about the pivot axis 15. The sensors and the control device, which were described above in connection with the exemplary embodiment of the prosthesis, are also accordingly present on the orthosis.

1. A method for controlling a prosthesis or orthosis of the lower extremity, the prosthesis or orthosis having an upper part (10) and a lower part (20) which is connected to the upper part (10) via a knee joint and is mounted so as to be pivotable relative to the upper part (10) about a joint axis (15), wherein an adjustable resistance device (40) is arranged between the upper part (10) and the lower part (20), by means of which resistance device (40) a resistance (Rf) is modified on the basis of sensor data, wherein state information is detected via sensors, a cyclical movement different than walking is determined, and the resistance (Rf) is adjusted to a low level during the cyclical movement, characterized in that the determination of the cyclical movement comprises the following steps:
a. detecting the flexion angle (α_{K}) and at least one absolute angle (α_{S}) of the lower part (20) and/or of the upper part (10) over at least one movement cycle,
b. identifying the cyclical movement from the relative movement of upper part (10) and lower part (20) and from the absolute movements of upper part (10) and/or lower part (20) in space.

2. The method as claimed in claim 1, characterized in that the reduction of the resistance (Rf) takes place only when a flexion angle change (ΣΔα_{K}), in particular the amount of the flexion angle change, added up over an interval in which certain conditions are met, is greater than a specified limit value, in particular greater than 240°.

3. The method as claimed in claim 1, characterized in that a cyclical movement is ascertained only if the flexion angle (α_{K}) is greater than a limit value, in particular greater than 10°, in particular greater than 15°.

4. The method as claimed in claim 1, characterized in that a cyclical movement is ascertained only if the angle of inclination (α_{S}) of the lower part (20) and/or the flexion angle (α_{K}) does not exceed and/or fall below certain limit values.

5. The method as claimed in claim 1, characterized in that a cyclical movement is ascertained only if the angle of inclination (α_{S}) of the lower part (20) forward relative to the vertical (G) is less than a limit value, in particular less than 5°.

6. The method as claimed in claim 1, characterized in that the end of the cyclical movement is identified from the fact that the lower part (20) is relieved of an axial force (FA) for a predetermined period of time and the axial force (FA) falls below a predetermined limit value and the flexion angle (α_{K}) falls below a limit value or the inclination (α_{S}) of the lower part (20) to the vertical (G) exceeds a limit value.

7. The method as claimed in claim 1, characterized in that, when the cyclical movement is interrupted while the lower part (20) is simultaneously subjected to an axial force (FA), an extension of the knee joint (1) below a specified flexion angle (α_{K}) is prevented and the flexion resistance (Rf) increased and, after an end stop is reached, followed by flexion and renewed extension, the resistance is set anew for the cyclical movement.

8. The method as claimed in claim 1, characterized in that, in order to determine the cyclical movement, the angular velocities of the flexion angle (α_{K}) and of the angle of inclination (α_{S}) are calculated, and the quotient of the angular velocities is determined in specified time segments.

9. The method as claimed in claim 8, characterized in that changes in the quotient of the angular velocities are determined as tangent slopes in specified time segments.

10. The method as claimed in claim 8, characterized in that the cyclical movement is ascertained when the time profile of the tangent slopes is increasing monotonically.

11. The method as claimed in claim 8, characterized in that an evaluation of the angular velocities takes place only when a limit value of the flexion angle velocity is exceeded.

12. The method as claimed in claim 1, characterized in that the phase space of two angle parameters and/or their derivatives is used to determine the cyclical movement, in particular the direction of rotation in the phase space is determined and used.

13. The method as claimed in claim 1, characterized in that the trajectory of the foot part (30) and/or its derivatives relative to a determined hip rotation point is determined from the knee angle (KA) and/or absolute angles of upper part (10) and lower part (20) and is used for the determination of the cyclical movement.
